(19) United States
(12) Patent Application Publication
(10) Pub. No.: US 2010/0172937 A1
(43) Pub. Date: Jul. 8, 2010

(54) ENVELOPED VIRUS NEUTRALIZING COMPOUNDS

(76) Inventor: Girish J. Kotwal, Louisville, KY (US)

Correspondence Address: STITES & HARBISON, PLLC, 400 W MARKET ST, SUITE 1800, LOUISVILLE, KY, 40202-3352, US

(21) Appl. No.: 12/598,702

(22) PCT Filed: May 2, 2008
(86) PCT No.: PCT/US08/62389

§ 371 Date: Feb. 8, 2010

Related U.S. Application Data
(60) Provisional application No. 60/915,782, filed on May 3, 2007.

(51) Int. Cl. A61K 31/194; A61K 36/185; A61K 39/12; A61K 39/21; A61K 39/215; A61K 39/245; A61K 39/145; A61K 39/29; A61K 39/285; A61P 31/12; A61P 31/16; A61P 31/18; A61P 31/22; A61P 31/20

(52) U.S. Cl. 424/209.1; 514/568; 424/777; 424/204.1; 424/208.1; 424/221.1; 424/229.1; 424/227.1; 424/228.1; 424/232.1

(57) ABSTRACT

Methods for reducing infectivity by providing an enveloped virus neutralizing compound (EVNC) are provided. Methods of preparing a vaccine, vaccine formulations, and methods of immunizing a subject a further provided. Methods of disinfecting a surface are still further provided. In some embodiments, compositions comprising an isolated and purified bioactive EVNC agent are provided.

### RELATED APPLICATIONS

The presently-disclosed subject matter claims the benefit of U.S. Provisional Patent Application Ser. No. 60/915,782, filed May 3, 2007; the disclosure of which is incorporated herein by reference in its entirety.

### TECHNICAL FIELD

The presently-disclosed subject matter relates to methods and compositions for reducing the infectivity of a virus. In particular, the presently-disclosed subject matter relates to contacting a virus particle with a bioactive enveloped virus neutralizing compound (EVNC) agent to reduce the infectivity of the virus particle.

### BACKGROUND

Enveloped viruses like human immunodeficiency virus (HIV), hepatitis B virus (HBV), hepatitis C virus (HCV), influenza, and herpes viruses together contribute annually to over a billion infections and significant mortality worldwide. HIV remains a major killer of young people in sub-Saharan Africa [1], the success of antiretrovirals (ARVs) and prevention education in the west has not been fully replicated in Africa and South Asia. With ARVs beyond the reach of the overwhelming majority of infected individuals in these regions, and with side effects (lactic acidosis, peripheral neuropathy and adipose deposition) difficult to manage, there is an urgent need for new therapeutic alternatives.

In addition to the many established challenges, Africa may soon have to deal with the spread of bird flu [2,3], which has emerged in the western part of the continent. Africa, with a large immunosuppressed and malnourished population, must be considered particularly vulnerable to this threat. Asia and South America are only slightly better off but would also be devastated by a pandemic flu. The few effective treatments of acute influenza include oseltamivir phosphate (TAMIFLU®), a neuraminidase inhibitor [4], but its availability in Africa is very limited. Shikimic acid, one of the main starting materials of TAMIFLU®, is obtained from star anise, a cooking spice from a tree grown in China but other sources of shikimic acid are being explored and fairly high concentrations of shikimic acid are found in natural sources, e.g. pine, spruce and fir trees. In a recent article, however, additional problems related to the use of shikimic acid and its development, as well as problems related to the synthesis of TAMIFLU® and the use of other starting materials have been revealed [5].

Furthermore, any influenza outbreak is likely to precede the availability of protective vaccines by a long period. Clearly, there is an urgent need to investigate promising antivirals from all possible sources [6,7]. Coal-derived humic acids and oxyfulvic acid, which are complex mixtures formed during the decomposition of organic matter including naturally-found cellulose have been previously shown to have anti-HIV properties [8,9]. Complex molecules like lectins and mucins which can interfere with attachment of enveloped viruses also appear to have a broad spectrum antiviral activity against HIV and poxviruses [11, 12, 13]. As such, these agents have been predicted to be effective against influenza but their effectiveness remains to be proved and the agents may not be practical because of their molecular size and their interference with normal tissue functions.

Thus, to significantly reduce infectivity, there is an urgent need for alternate prophylactic and treatment approaches that can be easily delivered, are non-toxic, and are unaffected by the rapid mutation rates of viruses.

### SUMMARY

This Summary lists several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

In some embodiments of the presently-disclosed subject matter, a method for reducing the infectivity of a virus is provided. The method comprises, in some embodiments, providing a bioactive EVNC agent and contacting a virus particle with the bioactive agent to thereby reduce the infectivity of the virus particle.

In some embodiments of the presently-disclosed subject matter, a method of reducing the infectivity of a virus in a subject is provided. In some embodiments, the method comprises administering to the subject a bioactive EVNC agent that contacts a virus particle in the subject and thereby reduces the infectivity of the virus particle in the subject. In some embodiments, the bioactive agent is administered in an aerosol formulation or a liquid formulation to the subject. In some embodiments, the bioactive agent is administered orally, intranasally, or both to the subject. In some embodiments, the subject is a mammal or a bird and, in some embodiments, the subject is human.

The presently-disclosed subject matter further provides, in some embodiments, a method of preparing a vaccine against a virus. In some embodiments, the method for preparing a vaccine comprises providing a bioactive EVNC agent, contacting a virus particle with the bioactive EVNC agent to thereby render the virus particle non-infectious, and formulating a vaccine comprising the non-infectious virus particle. In some embodiments, the vaccine comprises a pharmaceutically-acceptable carrier. In some embodiments, the vaccine comprises an adjuvant.

The presently-disclosed subject matter still further provides, in some embodiments, a vaccine formulation. In some embodiments, the vaccine formulation comprises an antigenic component and a pharmaceutically-acceptable carrier, wherein the antigenic component comprises an intact non-infectious virus particle bound to a bioactive EVNC agent. In some embodiments, the vaccine comprises an adjuvant.

In some embodiments of the presently-disclosed subject matter, a method of immunizing a subject against a virus is provided. In some embodiments, the method of immunizing a subject against a virus comprises administering to the subject an effective amount of an immunogenic composition comprising a pharmaceutically-acceptable carrier and an intact non-infectious virus particle bound to a bioactive EVNC agent. In some embodiments, the subject is a mammal or a bird. In some embodiments, the subject is human. Further, in some embodiments the immunogenic composition comprises an adjuvant.

The presently-disclosed subject matter further provides, in some embodiments, a method of disinfecting a surface. In some embodiments, the disinfecting method comprises providing a disinfectant composition, comprising a bioactive EVNC agent, and contacting the surface with the disinfectant composition for a time period sufficient to reduce infectivity of a virus on the surface and thereby disinfect the surface. In some embodiments of the disinfecting method, the virus particle is rendered non-infectious after contacting the virus-particle with the bioactive agent.

In some embodiments of the presently-disclosed methods and formulations, the bioactive agent can comprise at least one peak from a high pressure liquid chromatography (HPLC)/mass spectrometry pattern of FIG. 1, one or more mass spectrometry spectra of FIGS. 2-4, or both. In some embodiments, the bioactive agent comprises a fraction eluted with an elution buffer from a hydrophilic-lipophilic balance (HLB) column, wherein the elution buffer comprises methanol at a concentration of from about 75% v/v to about 95% v/v. from about 80% v/v to about 90% v/v, or, in some embodiments, about 80%.

In some embodiments of the presently-disclosed methods and formulations, the bioactive agent has a molecular weight of about 500 Da or less. In some embodiments, the bioactive agent has a molecular weight of about 200 Da to about 500 Da. In some embodiments, the bioactive agent has a molecular weight of about 226 Da, about 340 Da, about 411 Da, about 454 Da, about 450 Da, or about 472 Da. Further, in some embodiments, the bioactive agent comprises fulvic acid, pomegranate juice, or fractions thereof. Still further, in some embodiments, the bioactive agent is het stable and exhibits antiviral biological activity after a heat treatment of about 121° C.

The presently-disclosed subject matter further provides, in some embodiments of the presently-disclosed methods and formulations, the virus particle contacted/treated is an enveloped virus. In some embodiments, the virus particle is a virus selected from the group consisting of human immunodeficiency virus (HIV), corona virus, herpes virus, influenza virus, hepatitis B virus (HBV), hepatitis C virus (HCV), vaccinia virus, Chikangunya virus, and variola virus. In some embodiments of the presently-disclosed methods and formulations, the virus particle is prevented from attaching to a host cell, entering a host cell, or both.

In some embodiments of the presently-disclosed subject matter a composition comprising an isolated and purified bioactive EVNC agent is provided, wherein the bioactive agent comprises at least one peak from a high pressure liquid chromatography (HPLC)/mass spectrometry pattern of FIG. 1, one or more mass spectrometry spectra of FIGS. 2-4, or both. In some embodiments of the EVNC compositions, the bioactive agent comprises a fraction eluted with an elution buffer from a hydrophilic-lipophilic balance (HLB) column, wherein the elution buffer comprises methanol at a concentration of from about 75% v/v to about 95% v/v. In some embodiments, the elution buffer comprises methanol at a concentration of from about 80% v/v to about 90% v/v. Further, in some embodiments of the composition, the elution buffer comprises methanol at a concentration of about 80%.

Still further, in some embodiments of the EVNC compositions, the bioactive agent has a molecular weight of about 500 Da or less. In some embodiments, the bioactive agent has a molecular weight of about 200 Da to about 500 Da, and, in some embodiments, the bioactive agent has a molecular weight of about 226 Da, about 340 Da, about 411 Da, about 454 Da, about 450 Da, and/or about 472 Da. In some embodiments, an EVNC composition is further provided wherein the bioactive agent comprises fulvic acid, pomegranate juice, or fractions thereof. In some embodiments, the bioactive agent exhibits antiviral biological activity after a heat treatment of about 121° C.

Accordingly, it is an object of the presently-disclosed subject matter to provide methods of reducing infectivity as well as vaccines and compositions for reducing infectivity of viruses. This object is achieved in whole or in part by the presently-disclosed subject matter.

An object of the presently-disclosed subject matter having been stated hereinabove, and which is achieved in whole or in part by the presently-disclosed subject matter, other objects and advantages will become evident to those of ordinary skill in the art after a study of the following description of the presently-disclosed subject matter, Figures, and non-limiting Examples.

### BRIEF DESCRIPTION OF THE DRAWINGS

FIG. 1 is a mass spectrometry profile of compounds from fulvic acid that weigh less than 3000 Da and were separated by HPLC using a C18 column. The peaks correspond to various bioactive enveloped virus neutralizing compounds, some with approximate molecular weights of about 226, about 340, about 454, and about 472.

FIG. 2 is a composite NMR profile of fractions of fulvic acid that are eluted from Oasis HLB columns with 5%, 10%, 20%, 40%, 80%, and 100% methanol.

FIG. 3 is a composite NMR profile of fractions of pomegranate juice that are eluted from an HLB column with 5%, 10%, and 20% methanol.

FIG. 4 is a portion of the composite NMR profile of FIG. 3 showing fractions of pomegranate juice that are eluted from an HLB column with 5%, 10%, and 20% methanol.

FIG. 5 is an illustration of potential mechanisms of action of EVNCs.

FIG. 6 is a photograph of infectivity and hemaglutinin assays with fulvic acid and pomegranate juice.

FIG. 7 is a graph showing weight loss as a function of time in mice intranasally administered a normally highly lethal dose of influenza virus that had been treated with fulvic acid.

### DETAILED DESCRIPTION

Unless defined otherwise, all technical and scientific terms used herein have the same meaning as commonly understood to one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” or “a virus” includes a plurality of such cells or viruses, respectively, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

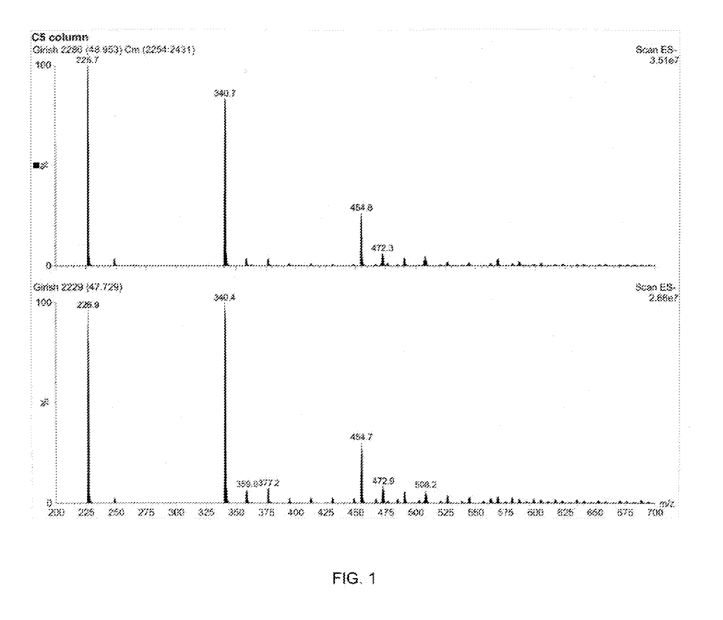

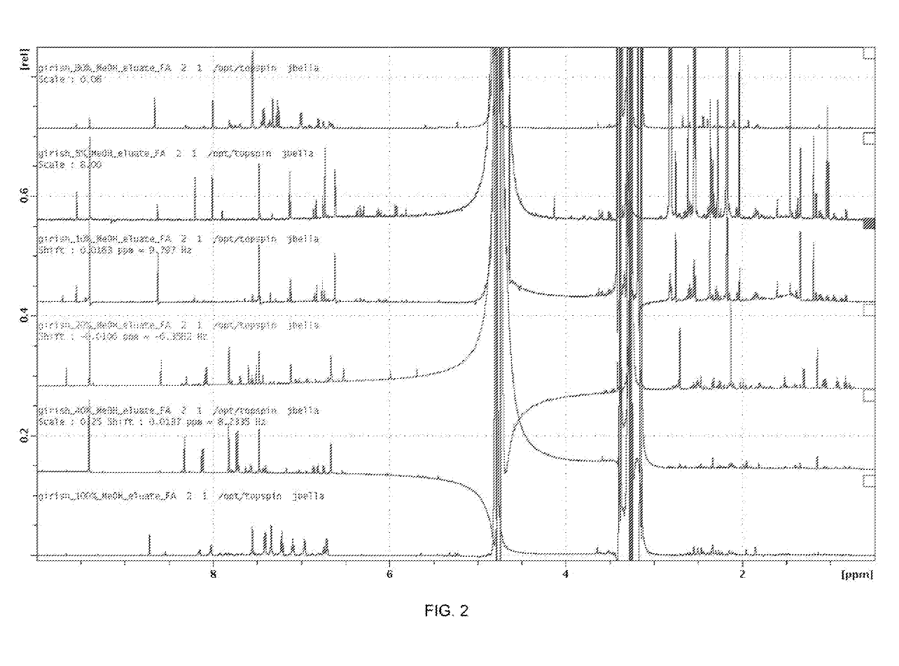

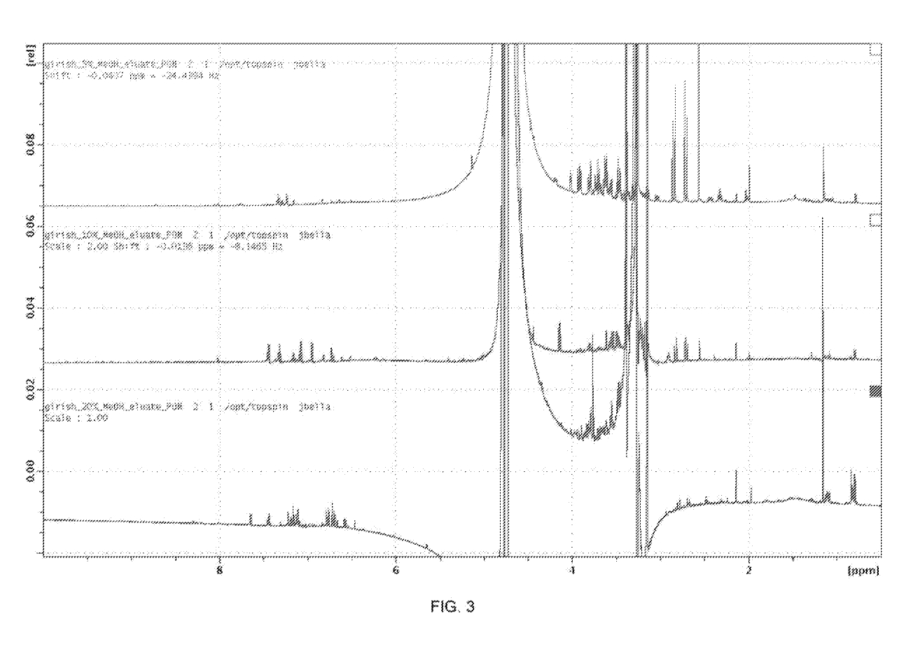

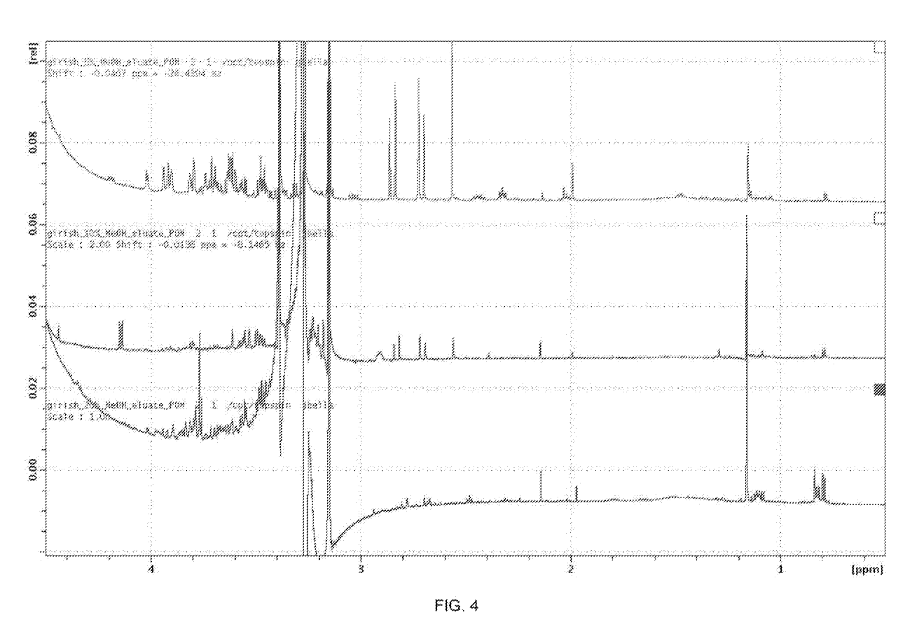

The presently-disclosed subject matter provides an isolated and purified bioactive agent, which is an enveloped virus neutralizing compound (EVNC). In some embodiments, the EVNC bioactive agent comprises at least one peak from a high pressure liquid chromatography (HPLC)/mass spectrometry pattern of FIG. 1, one or more mass spectrometry spectra of FIGS. 2-4, or both.

EVNCs are novel heat stable (to 121° C.) compounds that can be acidic and can be isolated by fractionation (e.g., by high pressure liquid chromatography (HPLC)) of plant materials, such as fulvic acid and/or pomegranate juice components, and identification of EVNCs within particular fractions, or chemically-synthesized equivalents of the bioactive compounds therein. Fulvic acid is a complex mixture of plant acids resulting from oxidation of plant carbohydrates and can be derived from any cellulose containing plant material. In some embodiments, the starting fulvic acid plant material can be derived from plant carbohydrates including bituminous coal or materials from living plants.

In some embodiments, the EVNC can be produced from plant material which can be induced from leaf-cultures and grown in liquid culture in nutrient bioreactors using methods generally understood by those of skill in the art. Base plant extract derived from the bioreactors can then be filtered and otherwise treated to further purify the bioactive components of the extract. In some embodiments, the plant extract is filtered through a 1000 Da filter, and in some embodiments, the plant extract is filtered through a 500 Da filter. In other embodiments, the bioactive agent is an EVNC, which has been chemically-synthesized or extracted from one or more natural sources.

In some embodiments, the bioactive EVNC can be acidic. In some embodiments, the bioactive EVNC has a molecular weight of about 500 Da or less. In some embodiments, the bioactive EVNC has a molecular weight of about 400 Da to about 500 Da, and in some embodiments the bioactive EVNC has a molecular weight of about 411 Da. In some embodiments, the bioactive EVNC has a molecular weight of about 226 Da, about 340 Da, about 411 Da, about 454 Da, about 450 Da, or about 472 Da. In some embodiments, the bioactive EVNC comprises fulvic acid, either isolated from a natural source such as a plant material or chemically-synthesized. In some embodiments, the bioactive EVNC is isolated from pomegranate juice. Further, in some embodiments, the bioactive agent exhibits antiviral biological activity after a heat treatment of about 121° C.

The EVNCs disclosed herein exhibit antiviral activity and have been shown to be non-toxic and non-mutagenic. Without wishing to be bound by theory, the presently-disclosed subject matter indicates that the bioactive EVNCs can render a target virus non-infectious by inhibiting entry into host cells, possibly by binding to sugar chains on one or more viral surface glycoproteins, and thereby serving as an “entry inhibitor” (i.e., reducing or preventing attachment to a host cell and/or entry into a host cell by the virus).

“Virus particle” and “virus” are used interchangeably herein and refer to any of various submicroscopic parasites of plants, animals, and bacteria that often cause disease and that comprise a core of RNA or DNA surrounded by a protein coat, and in some viruses a lipid-based envelope surrounding the protein coat. Viruses are unable to replicate without a host cell and are essentially inert in an extracellular environment. A virus attaches to a host cell via interaction with specific receptor molecules on the host cell and on the virus, which trigger entry of the virus into the host cell. Once inside the host cell many viruses conscript, in whole or in part (depending on the virus), host cell transcription and translation machinery in order to replicate. The replicated virus is then released from the cell to continue the infection of additional host cells. “Virus particle” generally refers to an individual virion particle, whereas “virus” can more generally refer to one or more virion particles, but the terms are not intended to be limited so, and each may refer to one or more virion particles. Thus, in some embodiments, the virus to which the bioactive agent exhibits antiviral activity can be a non-enveloped or an enveloped virus. Further, the bioactive agent can have broad antiviral activity against multiple taxonomically distinct virus families. In particular embodiments, the bioactive agent exhibits antiviral activity against enveloped viruses, including but not limited to human immunodeficiency virus (HIV), corona virus, herpes virus (e.g., HSV-1 and HSV-2), influenza virus (e.g., H5N1, H3N2, H1N1, etc.), hepatitis B virus (HBV), hepatitis C virus (HCV), vaccinia virus, Chikangunya virus, and variola virus.

“Antiviral activity”, as the term is used herein, generally refers to destruction or sequestration (e.g., coating of the virus through binding of the antiviral agent to the virus) of a virus resulting in inhibition of the growth and/or reproduction of viruses in a host cell or organism and/or reduction in the ability of the virus to infect a host (i.e., “infectivity of the virus”). For example, bioactive EVNCs disclosed herein can bind to a virus particle, reducing or eliminating the ability of the virus particle to attach to and/or enter a host cell, thereby rendering the virus particle non-infective and effectively neutralizing it, since a virus cannot replicate outside of a host cell. A virus particle trapped within an extracellular milieu of the host can be more susceptible to destruction by the host immune system than an intracellular viral particle.

Antiviral activity of a bioactive agent can be measured in vivo as a decrease in viral load (e.g., the number of viral particles in the blood of the host) over time or as a decrease in symptoms within the host associated with viral infection after treatment with the bioactive agent. Antiviral activity of a bioactive agent can also be measured in vitro utilizing, for example, cell culture assays. For example, in some embodiments of the presently-disclosed subject matter, a cell line (e.g., an African green monkey kidney (BSC-1) cell line) is cultivated as a monolayer in a culture dish and a virus known to be cytolytic for the cell line (e.g., vaccinia virus) added to the culture media either in the presence (test sample) or absence (negative control sample) of a test bioactive agent. In some instances, the test bioactive agent and virus can be mixed together for a period of time prior to addition to the culture media. A decrease in cell lysis by the virus in the presence of the test bioactive agent as compared to the negative control is indicative of antiviral activity by the test bioactive agent. Potency of the bioactive agent can be correlated to the level of inhibition of lysis by the virus.

In some embodiments, the bioactive EVNC is further purified to remove contaminants and unnecessary constituents from the base composition (e.g., pomegranate juice or fulvic acid (e.g., derived from plant materials)) by any of a number of purification techniques known in the art, including but not limited to high performance liquid chromatography (HPLC), dialysis, conventional column chromatography, hydrophilic-lipophilic balance (HLB) columns, ultrafiltration and other methods. Alternatively, the bioactive EVNC can be a chemically-synthesized composition matching the chemical characteristics of an EVNC derived from a natural source, such as but not limited to fulvic acid and pomegranate juice.

Although not required to provide the desired anti-viral activity, further purification can provide certain advantages. For example, removal of substituents of plant extracts not required for anti-viral activity can increase the bioactivity of the extract by concentrating the active components and removing potentially interfering components. As one non-limiting example, further purification of the pomegranate juice or fulvic acid can remove unnecessary components that could potentially cause toxicity in subjects administered the unpurified plant extracts, including for example cytotoxic peptides and heavy metals such as mercury, iron, arsenic and lead.

In some embodiments, the EVNCs are generated through purification by elution through an HPLC column of pomegranate juice or fulvic acid (e.g., derived from plant extract), and one or more eluted fractions exhibiting anti-viral activity are collected for use as the purified bioactive agent. HPLC separation techniques are generally known to those of skill in the art and an appropriate HPLC stationary phase material, mobile phase material and elution conditions can be chosen based on desired exclusion or inclusion parameters of the selected fractions.

In a particular embodiment, a C18 column can be utilized with a 1 mL loop. Methanol is passed through the column as an elution buffer and the concentration varied over time. In a particular embodiment, the methanol concentration is increased from 0% to 30% over 30 minutes at a flow rate of 1 ml/minute. Thirty fractions are then collected over the methanol concentration gradient, and an additional five fractions collected at 30% methanol concentration. In a particular non-limiting exemplary embodiment, a base plant extract is purified through the HPLC column as described immediately preceding, and 30 fractions collected over the methanol gradient of 0-30% and assayed for antiviral activity. The fraction or fractions exhibiting antiviral activity are then selected as purified bioactive agent.

In some embodiments, the bioactive agent elutes from a C18 column at a methanol concentration of between about 15% to about 25%, and in some embodiments about 20%. In some embodiments, the bioactive EVNC comprises at least one peak from a high pressure liquid chromatography (HPLC)/mass spectrometry pattern of FIG. 1, one or more mass spectrometry spectra of FIGS. 2-4, or both.

In some embodiments, the bioactive EVNC is further purified by elution through an HLB column and one or more eluted fractions exhibiting anti-viral activity are collected for use as a purified bioactive EVNC. HLB columns can also be used to further purify the base fulvic acid or pomegranate juice. In some embodiments, methanol extractions of 2.5% to 100% can be run through the HLB column containing base plant extract and fractions across the methanol concentration spectrum collected. The fractions can be tested for antiviral activity and the one or more fractions exhibiting antiviral activity selected as purified bioactive agent. In some embodiments, the bioactive EVNC comprises one or more fractions eluted from the HLB column with an elution buffer having a methanol concentration of from about 75% v/v to about 95% v/v, in some embodiments, from about 80% v/v to about 90% v/v, and in some embodiments about 80%.

In some embodiments, the presently-disclosed subject matter provides a method of reducing the infectivity of a virus. In some embodiments, the method comprises providing a bioactive EVNC, such as for example EVNCs isolated from pomegranate juice or fulvic acid, or a chemically-synthesized equivalent, and contacting a virus particle with the bioactive EVNC. The contacting of the virus particle with the bioactive EVNC can reduce the infectivity of the virus particle. In some embodiments, reducing the infectivity of the virus particle comprises preventing attachment of the virus particle to a host cell, entry of the virus particle into a host cell, or both.

In some embodiments, the presently-disclosed subject matter provides a method of reducing the infectivity of a virus in a subject. In some embodiments, the method comprises administering to the subject a bioactive EVNC, such as for example EVNC isolated from pomegranate juice or fulvic acid (e.g., derived from plant materials), or a chemically-synthesized equivalent, wherein the bioactive EVNC contacts a virus particle in the subject and thereby reduces the infectivity of the virus particle in the subject. In some embodiments of the method, reducing the infectivity of the virus particle comprises preventing attachment of the virus particle to a host cell, entry of the virus particle into a host cell, or both. In some embodiments of the method, the virus is an influenza virus and the bioactive agent is administered to the subject to prevent or reduce infection (e.g., lessen severity of symptoms or duration of illness

Further with respect to the therapeutic methods of the presently-disclosed subject matter, a preferred subject is a vertebrate subject. A preferred vertebrate is warm-blooded; a preferred warm-blooded vertebrate is a mammal. A preferred mammal is most preferably a human. As used herein, the term “subject” includes both human and animal subjects. Thus, veterinary therapeutic uses are provided in accordance with the presently-disclosed subject matter.

As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

Suitable methods for administering to a subject a bioactive EVNC in accordance with the methods of the present subject matter include but are not limited to systemic administration, parenteral administration (including intravascular, intramuscular, intraarterial administration), intranasal delivery, oral delivery, buccal delivery, subcutaneous administration, inhalation, intratracheal installation, surgical implantation, transdermal delivery, local injection, topical, and hyper-velocity injection/bombardment. Where applicable, continuous infusion can enhance drug accumulation at a target site (see, e.g., U.S. Pat. No. 6,180,082, incorporated herein by reference in its entirety).

The particular mode of administration used in accordance with the methods of the present subject matter depends on various factors, including but not limited to the bioactive EVNC and/or carrier employed, the severity of the condition to be treated and/or prevented, and mechanisms for metabolism or removal of the bioactive EVNC following administration. However, in a particular embodiment, the bioactive EVNC is administered in a liquid spray or drop or an aerosol formulation to the subject orally, intranasally, or both. In some embodiments, the formulations are delivered using atomizers or nebulizers. In some embodiments, the bioactive EVNC is formulated for administration in dry fogs for broad dispersion over particular areas, including for example rooms or hospital wards.

For administration of a therapeutic composition as disclosed herein, conventional methods of extrapolating human dosage based on doses administered to a murine animal model can be carried out using the conversion factor for converting the mouse dosage to human dosage: Dose Human per kg=Dose Mouse per kg×12 (Freireich et al., (1966) Cancer Chemother Rep. 50:219-244). Drug doses can also be given in milligrams per square meter of body surface area because this method, rather than body weight, achieves a good correlation to certain metabolic and excretionary functions. Moreover, body surface area can be used as a common denominator for drug dosage in adults and children as well as in different animal species as described by Freireich et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m².

For oral administration, a satisfactory result can be obtained employing the bioactive EVNC, such as for example EVNCs isolated from pomegranate juice or fulvic acid, or a chemically-synthesized equivalent, in an amount ranging from about 0.01 mg/kg to about 100 mg/kg and preferably from about 0.1 mg/kg to about 30 mg/kg. A preferred oral dosage form, such as liquid suspensions, will contain the bioactive agent in an amount ranging from about 0.1 to about 500 mg, preferably from about 2 to about 50 mg, and more preferably from about 10 to about 25 mg.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, N.J.; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Fla.; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pa.; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

The formulations administered to the subject can take such forms as aerosols, suspensions, solutions or emulsions in oily or aqueous vehicles, and can contain formulatory agents such as suspending, stabilizing and/or dispersing agents. Alternatively, the bioactive EVNC can be in powder form for constitution with a suitable vehicle, e.g., sterile pyrogen-free water, before use. Further, in some embodiments, the bioactive EVNC can be formulated for impregnation in clothing or barrier material, such as for example masks, gloves, surgical gowns, condoms, etc.

The formulations can be presented in unit-dose or multi-dose containers, for example sealed ampoules and vials, and can be stored in a frozen or freeze-dried (lyophilized) condition requiring only the addition of sterile liquid carrier immediately prior to use.

In some embodiments, the bioactive EVNC can be prepared in pharmaceutical compositions formulated for topical administration, for instance, in a cream, a paste, a gel, a foam, an ointment, a spray, a lubricant, an emulsion or suspension. For example, the topical administration can include administration in, around or on the genitalia, the genito urinary tract and/or the rectum. Thus, the topical administration can comprise intravaginal administration, penile administration, or rectal administration of the bioactive EVNC to aid in the prevention or reduction of risk of infectivity by a virus (e.g., HIV) during, for example, sexual contact.

For oral administration, the compositions can take the form of, for example, tablets or capsules prepared by a conventional technique with pharmaceutically-acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated by methods known in the art.

Liquid preparations for oral or intranasal administration can take the form of, for example, aerosols, solutions, syrups or suspensions, or they can be presented as a dry product for constitution with water or other suitable vehicle before use. Such liquid preparations can be prepared by conventional techniques with pharmaceutically-acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner.

The bioactive EVNC can also be formulated as a preparation for implantation or injection. Thus, for example, the bioactive EVNC can be formulated with suitable polymeric or hydrophobic materials (e.g., as an emulsion in an acceptable oil) or ion exchange resins, or as sparingly soluble derivatives (e.g., as a sparingly soluble salt).

The bioactive EVNC can also be formulated in rectal compositions (e.g., suppositories or retention enemas containing conventional suppository bases such as cocoa butter or other glycerides), creams or lotions, or transdermal patches.

In some embodiments of the presently-disclosed subject matter, a method of preparing a vaccine against a virus is provided. In some embodiments, the method comprises providing a bioactive EVNC, such as for example EVNCs isolated from pomegranate juice or fulvic acid (e.g., derived from plant materials), or a chemically-synthesized equivalent; contacting a virus particle with the bioactive agent, thereby rendering the virus particle non-infectious; and formulating a vaccine comprising the non-infectious virus particle. In some embodiments, rendering the virus particle non-infectious comprises preventing attachment of the virus particle to a host cell, entry of the virus particle into a host cell, or both.

Further, in some embodiments of the presently-disclosed subject matter, a vaccine formulation is provided. In some embodiments, the vaccine formulation comprises an antigenic component and a pharmaceutically-acceptable carrier, wherein the antigenic component comprises an intact non-infectious virus particle bound to a bioactive EVNC, such as for example EVNCs isolated from pomegranate juice or fulvic acid (e.g., derived from plant materials), or a chemically-synthesized equivalent. In some embodiments, the non-infectious virus particle is a virus particle that does not attach to a host cell, enter a host cell, or both. In some embodiments, the vaccine further comprises an adjuvant.

Still further, in some embodiments of the presently-disclosed subject matter, a method of immunizing a subject against a virus is provided. In some embodiments, the method comprises administering to the subject an effective amount of an immunogenic composition comprising a pharmaceutically-acceptable carrier and an intact non-infectious virus particle bound to a bioactive EVNC, such as for example EVNCs isolated from pomegranate juice or fulvic acid (e.g., derived from plant materials), or a chemically-synthesized equivalent. As used herein, the term “effective amount” means a dosage sufficient to provide immunization against the virus. This amount can vary depending on the subject and the virus being vaccinated against. Determination and adjustment of an effective dose, as well as evaluation of when and how to make such adjustments, are known to those of ordinary skill in the art. In some embodiments, the immunogenic composition further comprises an adjuvant. In some embodiments, the non-infectious virus particle is a virus particle that does not attach to a host cell, enter a host cell, or both.

In one embodiment, the vaccine formulations of the presently-disclosed subject matter include an effective immunizing amount of one or more of the above-described viruses bound to a bioactive EVNC, and particularly in some embodiments, one or more enveloped viruses. Purified or unpurified viruses can be used directly in a vaccine composition disclosed herein once the viruses have been inactivated by contact with a bioactive agent of the presently-disclosed subject matter. Thus, the presently-disclosed subject matter provides for preparation of an immunogenic viral vaccine without requiring additional chemical inactivation (which can destroy immunogenicity of the virus) or multiple serial passages of the virus to select for attenuated strains.

The term “immunogenic composition” as used herein refers to any composition able, once it has been administered to a subject, e.g., a mammal or bird, to elicit a protective immune response against the parasite or antigen or immunogen or epitope. The term “vaccine” as used herein refers to a formulation which, once it is administered, prevents or reduces the risk of infection or which ameliorates the symptoms of infection. The protective effects of a vaccine formulation against a pathogen are normally achieved by inducing in the subject an immune response, either a cell-mediated or a humoral immune response or a combination of both. Generally speaking, abolished or reduced incidences of viral infection, amelioration of the symptoms, or accelerated elimination of the viruses from the infected subjects are indicative of the protective immunogenic effects of a vaccine formulation. The vaccine formulations of the present subject matter provide protective effects against infections caused by viruses disclosed hereinabove, including for example a wide variety of enveloped viruses.

Typically, a vaccine contains between about 1×10⁶ and about 1×10⁸ virus particles, with a pharmaceutically-acceptable carrier and in some instances an adjuvant, in a volume of between about 0.5 and 5 ml. The precise amount of a virus in a vaccine composition effective to provide a protective effect can be determined by a skilled clinician. Pharmaceutically-acceptable carriers suitable for use in vaccine compositions can be any of those described herein.

The immunogenic compositions and vaccines according to the presently-disclosed subject matter can comprise or consist essentially of one or more adjuvants. Suitable adjuvants for use in the practice of the present subject matter include, but are not limited to (1) polymers of acrylic or methacrylic acid, maleic anhydride and alkenyl derivative polymers, (2) immunostimulating sequences (ISS), such as oligodeoxyribonucleotide sequences having one or more non-methylated CpG units (Klinman et al., Proc. Natl. Acad. Sci., USA, 1996, 93, 2879-2883; WO98/16247), (3) an oil in water emulsion, such as the SPT emulsion described on p 147 of “Vaccine Design, The Subunit and Adjuvant Approach” published by M. Powell, M. Newman, Plenum Press 1995, and the emulsion MF59 described on p 183 of the same work, (4) cation lipids containing a quaternary ammonium salt, (5) cytokines, (6) aluminum hydroxide or aluminum phosphate or (7) other adjuvants discussed in any document cited and incorporated by reference into the instant application, or (8) any combinations or mixtures thereof.

The oil in water emulsion (3), which can be particularly appropriate for viral vaccines, can be based on: light liquid paraffin oil (European pharmacopoeia type), isoprenoid oil such as squalane, squalene, oil resulting from the oligomerization of alkenes, e.g. isobutene or decene, esters of acids or alcohols having a straight-chain alkyl group, such as vegetable oils, ethyl oleate, propylene glycol, di(caprylate/caprate), glycerol tri(caprylate/caprate) and propylene glycol dioleate, or esters of branched, fatty alcohols or acids, especially isostearic acid esters. The oil can be used in combination with emulsifiers to form an emulsion. The emulsifiers can be nonionic surfactants, such as: esters of, on the one hand, sorbitan, mannide (e.g. anhydromannitol oleate), glycerol, polyglycerol or propylene glycol and, on the other hand, oleic, isostearic, ricinoleic or hydroxystearic acids, the esters being optionally ethoxylated, or polyoxypropylene-polyoxyethylene copolymer blocks, such as Pluronic, e.g., L121.

Among the type (1) adjuvant polymers, preference is given to polymers of crosslinked acrylic or methacrylic acid, especially crosslinked by polyalkenyl ethers of sugars or polyalcohols. These compounds are known under the name carbomer (Pharmeuropa, vol. 8, no. 2, June 1996). One skilled in the art can also refer to U.S. Pat. No. 2,909,462, which provides such acrylic polymers crosslinked by a polyhydroxyl compound having at least three hydroxyl groups, preferably no more than eight such groups, the hydrogen atoms of at least three hydroxyl groups being replaced by unsaturated, aliphatic radicals having at least two carbon atoms. The preferred radicals are those containing 2 to 4 carbon atoms, e.g. vinyls, allyls and other ethylenically unsaturated groups. The unsaturated radicals can also contain other substituents, such as methyl. Products sold under the name CARBOPOL™ (BF Goodrich, Ohio, USA) are especially suitable. They are crosslinked by allyl saccharose or by allyl pentaerythritol. Among them, reference is made to CARBOPOL™ 974P, 934P and 971 P.

As to the maleic anhydride-alkenyl derivative copolymers, preference is given to EMA (Monsanto), which are straight-chain or crosslinked ethylene-maleic anhydride copolymers and they are, for example, crosslinked by divinyl ether. Reference is also made to J. Fields et al., Nature 186: 778-780, Jun. 4, 1960.

Immunization protocols can be optimized using procedures generally known in the art. A single dose can be administered to a subject, or alternatively, two or more inoculations can take place with intervals of several weeks to several months. The extent and nature of the immune responses induced in the subject can be assessed by using a variety of techniques. For example, sera can be collected from the inoculated subject and tested for the presence of antibodies specific for viruses immunized against, e.g., in a conventional virus neutralization assay. Detection of responding CTLs in lymphoid tissues can be achieved by assays such as T cell proliferation, as indicative of the induction of a cellular immune response. The relevant techniques are well described in the art, e.g., Coligan et al. Current Protocols in Immunology, John Wiley & Sons Inc. (1994).

Formulations described herein can further comprise a pharmaceutically-acceptable carrier. Suitable formulations include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents. The pharmaceutically-acceptable carriers or vehicles or excipients are well known to the one skilled in the art. For example, a pharmaceutically-acceptable carrier or vehicle or excipient can be a 0.9% NaCl (e.g., saline) solution or a phosphate buffer. The pharmaceutically-acceptable carrier or vehicle or excipients can be any compound or combination of compounds facilitating the administration of the bioactive agent; advantageously, the carrier, vehicle or excipient can facilitate administration, delivery and/or improve preservation of the bioactive agent and/or non-infectious virus.

The presently-disclosed subject matter provides a method of disinfecting a surface. In some embodiments, the method comprises providing a disinfectant composition comprising a bioactive EVNC, such as for example EVNCs isolated from pomegranate juice or fulvic acid (e.g., derived from plant materials), or a chemically-synthesized equivalent; and contacting the surface with the disinfectant composition for a time period sufficient to reduce infectivity of a virus on the surface, thereby disinfecting the surface. The virus particle is rendered non-infectious after contacting the bioactive EVNC. In some embodiments, non-infectious virus particle is a virus particle that does not attach to a host cell, enter a host cell, or both. In some embodiments, the virus particle on the surface is rendered non-infectious after contact with the bioactive agent for 10 minutes or less, 5 minutes or less in some embodiments, and less than one minute in other embodiments.

### EXAMPLES

The presently-disclosed subject matter is further illustrated by the following specific but non-limiting examples. The following examples are prophetic, notwithstanding the numerical values, results and/or data referred to and contained in the examples. Further, the following examples may include compilations of data that are representative of data gathered at various times during the course of development and experimentation related to the present subject matter.

### Materials and Methods

Fulvic acid and Pomegranate juice: Fulvic acid was made using a bioreactor system that produces organic plants acids. These plant acids were non-toxic and non-mutagenic at a concentration that displays potent activity against a wide range of drug resistant organisms. The process commenced with a carbohydrate solution, comprised of either a cellulose, bituminous coal, mono-saccharide, di-saccharide, poly-saccharide, or starch solution that was introduced into a computerized bioreactor system under controlled wet oxidation, temperatures and pressures. Following an incubation period in the presence of oxygen, plant acids of varying length were extracted and filtered to a fraction containing fragments between 200 and 4000 Dalton [9,10]. Some pomegranate juice was obtained from commercially-available sources in the United States and is commonly called POM. It was filtered by bacteria proof filtration. Pomegranate juice was also prepared from pomegranates (Punica grantum) in South Africa, and had almost identical antiviral activity to POM when tested against vaccinia virus. This pomegranate juice was prepared by removing the seeds with the surrounding pulp and blending it through a blender with a filter to block the coarse material getting into the juice and then filtering by bacteria proof filtration and using this as the testing material.

Cell culture and virus stocks: MDCK cells were used to culture and titrate influenza viruses with and without treatment. The titration and hemagglutination assay was done by standard procedure [15].

HPLC/NMR Mass Spectrometry: Fulvic Acid or pomegranate juice was ultra-filtered with a 3000 Da cutoff filter for separation using Waters Oasis HLB method. The ultra-filtered fulvic acid or pomegranate juice was then loaded into a glass vial. 7 μl of the ultra-filtered fulvic acid or pomegranate juice was injected for HPLC analysis using a C18 column. The HLB column was treated with 2 ml of methanol and was then washed with 2 ml of HPLC grade distilled water. Upon loading of the sample, the unbound sample was collected. The material was then washed with 2 ml of HPLC grade distilled water. The material bound to the column was then sequentially eluted over a period of 30 minutes using a methanol gradient of 0% to 100% methanol. The eluate was then passed through a mass spectrometry negative ionization system. Exemplary mass spectrometry and composite NMR profiles of the fulvic acid and pomegranate juice compounds are shown in FIGS. 1-4.

### Results

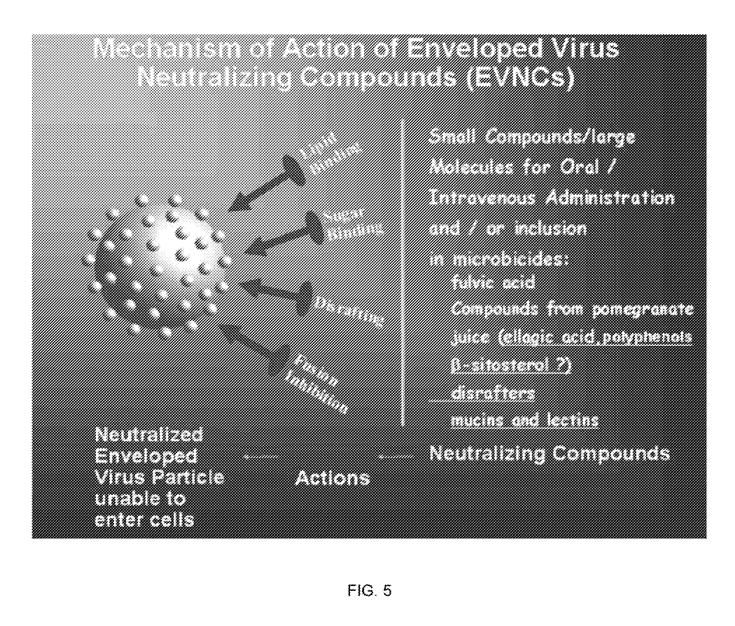

The bioactive fulvic acid was found to be in the range of 200 to 600 Da; with the peak activity observed around fractions corresponding to 113, 226, 341, and 452 Da indicating that the active compounds can be repeats of a monomeric unit of about 113 Da (FIG. 1). With regard to the mechanism of action and structure of fulvic acid and bioactive compounds from pomegranate juice, the hemagglutinating activity was inhibited indicating that fulvic acid blocks entry of the virus by interacting with the sugar or lipid moiety or both of the surface glycoproteins of enveloped viruses (FIG. 5). These studies indicate that the bioactive molecule renders the virus non-infectious by inhibiting entry into the cells. Once a virus enters a cell, the agent has no effect on viral replication. Short- and long-term toxicity studies in vitro and in vivo show that the bioactive agent is safe. Physical and chemical analyses show that the formulation is acidic (around pH 2) and heat stable (survives autoclaving), and that the levels of iron and arsenic are well below the permissible levels.

To confirm that the formulation containing fulvic acid or pomegranate juice can neutralize the infectivity of a wide range of genetically diverse strains of a given enveloped virus, by interacting with sugar chains on the viral surface protein and serving as an entry inhibitor, the formulation's activity against influenza A/HK/x31 (H3N2), influenza A/Vietnam/1203/04 (H5N1), and a reassortant x31 containing the NS gene segment of an H5N1 isolate was tested. The reassortant x31 was described before [15]. All viruses were inactivated in a dose-dependent manner when treated for 5 min at room temperature with the fulvic acid containing formulation or for the same period at 37 degree centigrade with pomegranate juice.

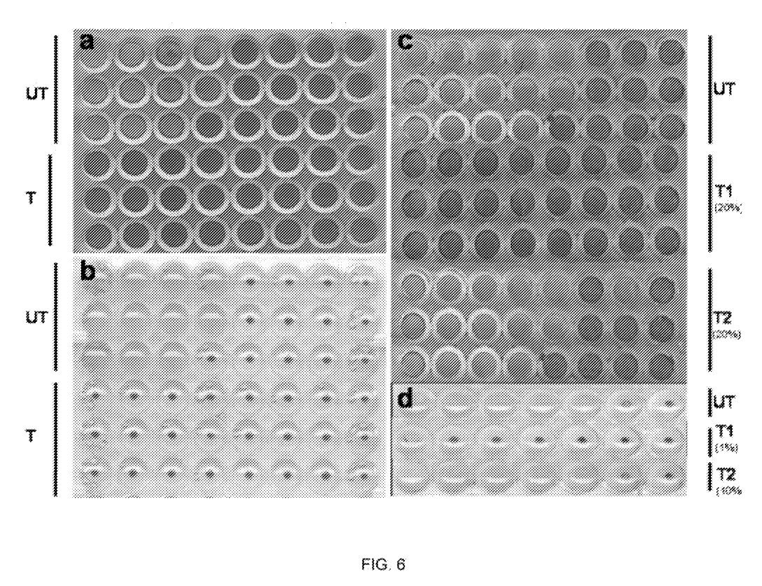

The effectiveness of the plant acid in neutralizing the infectivity of diverse influenza subtypes is shown in FIG. 6a-c. A pH-matched popular carbonated drink was included as a control in these experiments and had no effect on viral infectivity (FIG. 6c). Fulvic acid treatment of influenza A/x31 (H3N2) and A/Puerto Rico/8/34 (H1N1) resulted in a marked reduction in hemagglutination titer (FIG. 6d), supporting the idea that the bioactive agent may bind to the viral hemagglutinin and block attachment to cells.

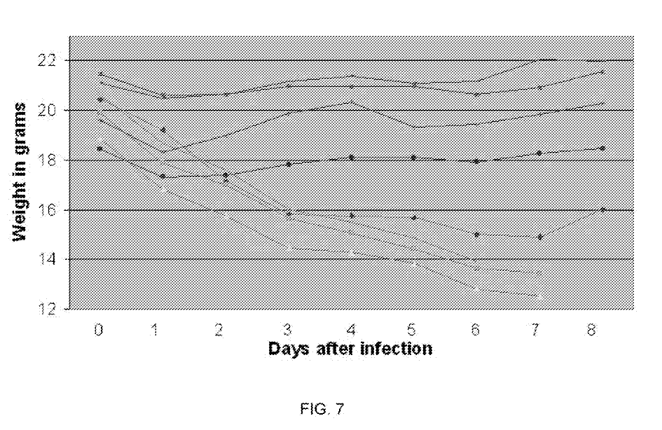

To obtain further evidence for the anti-viral action of fulvic acid, mice were intranasally administered a normally highly lethal dose of A/x31 that had been treated for 5 min with fulvic acid. Control mice received an equivalent dose of mock-treated virus. Mice that received the treated virus showed no signs of disease (FIG. 7, upper, dark shaded lines), whereas the mock-treated virus produced extensive weight loss (due to diminished water consumption) and 75% mortality (FIG. 7, lower, lightly shaded lines). In addition, the duration of the survival of the infected untreated mice mimics the duration of the period of survival from fever to death in H5N1 infected humans [16].

These studies indicate that compounds with antiviral activity from fulvic acid and from pomegranate juice neutralize the infectivity of diverse enveloped viruses and a number of subtypes of a given enveloped virus, thus indicating potential for their development as a treatment option that can be broadly effective against pandemic viruses like HIV, potentially pandemic viruses like influenza, and carcinogenic viruses like HBV and HCV. Further, carcinogenic viruses and potentially dangerous bioweapons like variola can be neutralized by the EVNCs. Demonstration of routine experimental neutralization of a live attenuated vaccinia virus, vGK5 [17] with the greatest structural robustness compared to all known enveloped viruses and similar to variola can be extrapolated to the carcinogenic hepatitis viruses and variola. These findings also indicate that certain plant acids, sugars and complex carbohydrates can have an application in the production of inactivated whole viral vaccines as well as microbicides. The EVNCs could be administered orally in concentrated capsules, intranasally as a nasal spray, or administered intravenously after extensive research into the bioavailability and proper well conceived clinical trials.

### REFERENCES

[1] G. J. Kotwal. HIV treatment and eradication in South Africa, J. R. Soc. Med. 2004; 97: 1-2.
[2] Palese P. Influenza: old and new threats. Nat. Med. Review. 2004; S82-87: 10 (12 Suppl).
[3] R. Webby, E. Hoffmann., R. Webster, Molecular constraints to interspecies transmission of viral pathogens, Nat. Med. 2004; 10 (12 Suppl):S77-81.
[4] E. De Clercq, Antiviral agents active against influenza A viruses, Nat Rev Drug discov 2006; 12:1015-1025.
[5] Farina, and Brown, J C. The supply problem. Angew Chem. Int. 2006; 45, 7330-7334
[6] D. Cyranoski, Threat of pandemic brings flu drug back to life, Nat. Med. 2005; 11:909.
[7] A. J. McCullers, The clinical need for new antiviral drugs directed against influenza virus, J. Infect. Dis. 2006; 193: 751-753.
[8] Van Rensburg, C. E. J., Dekker, J., Weis, R., Smith, T,-L, Rensburg, E. J, Schneider, J. Investigation of anti-HIV properties of oxihumate. Experimental Chemotherapy 2002; 48:138-143.
[9] Van Rensburg, A. van Straton, J. Dekker J. An in vitro investigation of the antimicrobial activity of oxifulvic acid. J. Antimicrob. chemother 2000; 46 853-854.
[10] Kotwal, G. J. et al. Anti-HIV, Anti-Poxvirus, and Anti-SARS Activity of a Nontoxic, Acidic Plant Extract from the Trifollium Species Secomet-V/anti-Vac Suggests That It Contains a Novel Broad-Spectrum Antiviral., Ann. NY Acad. Sci. 2005; 1056:293-302.
[11] Habte H H et al. (2006) The role of crude human saliva and purified salivary MUC5B and MUC7 mucins in the inhibition of human immunodeficiency virus type I in an inhibition assay. Virology Journal (Biomed Central) 3:99.
[12] Habte H H et al (2007) Antiviral activity of purified human breast milk mucin. Neonatology 92(2):96-104.
[13] Kaur A et al. (2007) Purification of 3 monomericmonocot mannose-binding lectins and their evaluation for anipoxyiralactivity: potential applications in multiple viral diseases caused by enveloped viruses. Biochemistry and Cell Biology 85(1):88-95.
[14] A. R. Neurath, N. Strick, Y. Y. Li and A. K. Debnath, Punica granatum (pomegranate) Juice provides an HIV-1 Entry Inhibitor and Candidate Topical Microbicide, Ann N Y Acad. Sci. 2005; 1056:311-327.
[15] A. S. Lipatov, S. Andreansky, R. J. Webby, D. J. Hutse, J. E. Rehg, S. Krauss, D. R. Perez, P. C. Doherty, R. G. Webster and M. Y. Sangster. Pathogenesis of Hong Kong H5N1 influenza virus NS gene reassortants in mice: the role of cytokines and B- and T-cell responses, J Gen Virol. 2005; 86:1121-1130.
[16] Yu, H. et al. The first confirmed human case of avian influenza A (H5N1) in Mainland China. Lancet 2006; 367: 84-85.
[17] Kotwal G J, Hugin A W, Moss B. Mapping and insertional mutagenesis of a vaccinia virus gene encoding a 13,800-Da secreted protein. Virology. 1989 August; 171(2):579-87.

It will be understood that various details of the presently-disclosed subject matter can be changed without departing from the scope of the subject matter. Furthermore, the foregoing description is for the purpose of illustration only, and not for the purpose of limitation.

1-13. (canceled)

14. A method of reducing the infectivity of a virus in a subject, the method comprising administering to the subject a bioactive EVNC agent, wherein the bioactive EVNC agent contacts a virus particle in the subject and thereby reduces the infectivity of the virus particle in the subject.

15-18. (canceled)

19. The method of claim 14, wherein the bioactive agent has a molecular weight of about 500 Da or less.

20-21. (canceled)

22. The method of claim 14, wherein the bioactive agent comprises fulvic acid, pomegranate juice, or fractions thereof.

23. The method of claim 14, wherein the bioactive agent exhibits antiviral biological activity after a heat treatment of about 121° C.

24. (canceled)

25. The method of claim 23, wherein the virus particle is a virus selected from the group consisting of human immunodeficiency virus (HIV), corona virus, herpes virus, influenza virus, hepatitis B virus (HBV), hepatitis C virus (HCV), vaccinia virus, Chikangunya virus, and variola virus.

26. The method of claim 14, wherein reducing the infectivity of the virus particle comprises preventing attachment of the virus particle to a host cell, entry of the virus particle into a host cell, or both.

27. The method of claim 14, wherein the bioactive agent is administered in an aerosol formulation or a liquid formulation to the subject.

28. The method of claim 26, wherein the bioactive agent is administered orally, intranasally, topically, or combinations thereof to the subject.

29. The method of claim 14, wherein the subject is a mammal or a bird.

30. The method of claim 28, wherein the subject is human.

31. A method of preparing a vaccine against a virus, the method comprising:
(a) providing a bioactive EVNC agent;
(b) contacting a virus particle with the bioactive EVNC agent, thereby rendering the virus particle non-infectious; and
(c) formulating a vaccine comprising the non-infectious virus particle.

32-35. (canceled)

36. The method of claim 31, wherein the bioactive agent has a molecular weight of about 500 Da or less.

37-38. (canceled)

39. The method of claim 31, wherein the bioactive agent comprises fulvic acid, pomegranate juice, or fractions thereof.

40. The method of claim 31, wherein the bioactive agent exhibits antiviral biological activity after a heat treatment of about 121° C.

41. The method of claim 31, wherein the virus particle is an enveloped virus.

42. The method of claim 41, wherein the virus particle is a virus selected from the group consisting of human immunodeficiency virus (HIV), corona virus, herpes virus, influenza virus, hepatitis B virus (HBV), hepatitis C virus (HCV), vaccinia virus, Chikangunya virus, and variola virus.

43. The method of claim 31, wherein rendering the virus particle non-infectious comprises preventing attachment of the virus particle to a host cell, entry of the virus particle into a host cell, or both.

44. The method of claim 31, wherein the vaccine comprises a pharmaceutically-acceptable carrier.

45. The method of claim 31, wherein the vaccine comprises an adjuvant.

46. A vaccine formulation, comprising an antigenic component and a pharmaceutically-acceptable carrier, wherein the antigenic component comprises an intact non-infectious virus particle bound to a bioactive EVNC agent.

47-50. (canceled)

51. The vaccine formulation of claim 46, wherein the bioactive agent has a molecular weight of about 500 Da or less.

52-53. (canceled)

54. The vaccine formulation of claim 46, wherein the bioactive agent comprises fulvic acid, pomegranate juice, or fractions thereof.

55. The vaccine formulation of claim 46, wherein the bioactive agent exhibits antiviral biological activity after a heat treatment of about 121° C.

56. The vaccine formulation of claim 46, wherein the virus particle is an enveloped virus.

57. The vaccine formulation of claim 56, wherein the virus particle is a virus selected from the group consisting of human immunodeficiency virus (HIV), corona virus, herpes virus, influenza virus, hepatitis B virus (HBV), hepatitis C virus (HCV), vaccinia virus, Chikangunya virus, and variola virus.

58. The vaccine formulation of claim 46, wherein the non-infectious virus particle is a virus particle that does not attach to a host cell, enter a host cell, or both.

59. The vaccine formulation of claim 46, wherein the vaccine comprises an adjuvant.

60-98. (canceled)
